(19) United States
(12) Patent Application Publication
(10) Pub. No.: US 2001/0029946 A1
(43) Pub. Date: Oct. 18, 2001

(54) Anesthesia ventilator with automatic control of the selected ventilation mode

(76) Inventor: Sebastien Kitten, Saulx Les Chartreux (FR)

Correspondence Address: YOUNG & THOMPSON, 745 SOUTH 23RD STREET 2ND FLOOR, ARLINGTON, VA, 22202

(21) Appl. No.: 09/777,909

(22) Filed: Feb. 7, 2001

(30) Foreign Application Priority Data
Feb. 7, 2000 (FR) 0001480

(51) Int. Cl. A61M 15/00; A61M 16/00; A62B 7/00; F16K 31/02; A62B 1/00

(52) U.S. Cl. 128/203.14; 128/204.21; 128/205.17

(57) ABSTRACT

An anesthesia apparatus of the type with several ventilation modes comprises a main gas circuit (1) formed by an inhalation branch (1a) and by an exhalation branch (1b), a gas accumulation member (4) whose internal volume varies and communicates with the main circuit (1), and relief valve means (6) which have an adjustable pressure set-point level and are arranged on the main circuit (1). In addition, detection means (11, 12, 13, 14) make it possible to determine an information item relating to the gas entering and/or leaving the accumulation member (4), information-processing means (10) make it possible to process said information item in order to deduce from it information concerning ventilation mode, and control means (15) act on the relief valve means (6) in order to automatically adjust the pressure set-point level of said relief valve means (6) as a function of the information concerning ventilation mode delivered by the processing means (10).

### BACKGROUND OF THE INVENTION

The present invention relates to the field of inhalation anesthesia and more particularly to an inhalation anesthesia apparatus comprising a gas circuit provided with means for monitoring, preferably permanently monitoring, the movements or flows of gas entering and leaving a gas accumulation reservoir, such as a reservoir balloon, provided on said gas circuit.

Anesthesia apparatuses traditionally comprise a closed system of gas channels or the like, also called the main circuit.

A main circuit is intended, on the one hand, to convey fresh anesthetic gases from a source of fresh anesthetic gases to the upper airways of the patient, and, on the other hand, to recover the gases exhaled by the patient, permit elimination of the carbon dioxide contained in these gases exhaled by the patient, and permit recycling and mixing of these purified exhaled gases with the fresh anesthetic gases before they are returned to the upper airways of the patient.

Such a circuit is therefore a closed system which functions as a loop and makes it possible to minimize the quantity of fresh anesthetic gas used to anesthetize the patient.

The loop circuit generally comprises an accumulation member, such as a gas reservoir balloon, intended to temporarily store at least some of the gases which have been exhaled by the patient and which have been purified by elimination of at least the greater part of the carbon dioxide which they contained.

The exhaled and purified gases are reused and returned to the patient during the subsequent inhalation phases, with possible addition of fresh anesthetic gases.

### DESCRIPTION OF THE PRIOR ART

Such devices have already been described many times in the prior art. For further details, reference can be made in particular to the documents EP-A-643978, U.S. Pat. No. 3,687,137, EP-A-292615 or EP-A-266964.

At present, existing inhalation anesthesia apparatuses function according to three primary modes of ventilation, namely spontaneous mode, manual mode, and controlled mode.

In spontaneous mode, the patient must be able to independently draw the quantity of respiratory gas he needs from the member accumulating the exhaled gases, for example a gas reservoir balloon.

In manual mode, it is the practitioner who controls the delivery of the gas contained in the accumulation member to the patient by exerting a pressure on the flexible wall of the accumulation member.

In controlled mode, also called mechanical mode, patient ventilation is completely automated, that is to say generated by the ventilator which ensures and controls the circulation of the gas between the patient and the gas accumulation member, and vice versa.

However, changing over from a mechanical or spontaneous ventilation mode to manual ventilation mode can presently only be done by manual switching of a relief valve by the practitioner, that is to say the physician or the like.

Thus, it will readily be appreciated that this poses a real safety problem.

This is because omitting to switch from manual mode to spontaneous mode, for example, forces the patient to fight against high gas pressures, generally pressures of 10 to 90 hPa, which can cause barotrauma to the patient on account of the rise in pressure throughout the ventilation circuit and, consequently, in the upper airways and lungs of said patient.

### SUMMARY OF THE INVENTION

The object of the present invention is therefore to make available an inhalation anesthesia apparatus and a method for operating such an apparatus, which apparatus and method make it possible to solve the abovementioned problems and thus minimize the risks to the patient.

In other words, the present invention aims to reduce the functioning of an anesthesia apparatus to just two ventilation modes, namely an assisted mode and a controlled mode, by virtue of the apparatus automatically recognizing the change-over between in particular the spontaneous mode and the manual mode, and by virtue of automatic reproduction of the switching operations triggered manually beforehand by the practitioner.

With the present invention it is therefore possible to simplify the use of anesthesia devices or machines and to increase patient safety.

The present invention thus concerns an anesthesia apparatus of the type with several ventilation modes, comprising at least:

a main gas circuit formed by at least one inhalation branch and by at least one exhalation branch,

a gas accumulation member whose internal volume is able to vary and which communicates with said main gas circuit,

relief valve means which have an adjustable pressure set-point level and are arranged on said main gas circuit,

detection means making it possible to determine at least one information item relating to the gas entering and/or leaving the accumulation member,

information-processing means making it possible to process said at least one information item relating to the gas entering and/or leaving in order to deduce from it at least one information item concerning ventilation mode, and

control means acting on the relief valve means in order to automatically adjust the pressure set-point level of said relief valve means as a function of at least the information concerning ventilation mode delivered by said information-processing means.

If appropriate, the apparatus according to the invention can have one or more of the following characteristics:

said detection means are arranged on or in proximity to the gas accumulation member;

said detection means are chosen from among:

the sensors for the flowrate of gas entering and/or leaving the gas accumulation member,

the sensors for the pressure of gas entering and/or leaving the gas accumulation member,

the switches arranged on or in proximity to the connection of the accumulation member and activated directly by the user when he wishes to actuate said relief valve means,

the mechanical transmitters making it possible to translate a variation in pressure in the accumulation member into at least one mechanical or electrical information item, and/or

the devices for analyzing the variations in the volume and/or shape and/or pressure of the gas accumulation member,

the gas accumulation member is a reservoir balloon;

the information-processing means comprise at least one microprocessor able to process the electronic information items transmitted by the detection means in order to deduce therefrom at least one information item concerning ventilation mode, that is to say the microprocessor allows the ventilation mode imposed by the operator to be taken into account and ensures electronic control of the ventilation means;

the control means act on the relief valve means in order to automatically reduce the pressure set-point level of said relief valve means when the information concerning ventilation mode delivered by the information-processing means corresponds to information on spontaneous ventilation mode;

the control means act on the relief valve means in order to automatically increase the pressure set-point level of said relief valve means when the information concerning ventilation mode delivered by the information-processing means corresponds to information on manual ventilation mode;

the gas accumulation member and/or the relief valve means are arranged on the exhalation branch or on the inhalation branch;

detection means are arranged on a connection line linking the gas accumulation member to the main gas circuit.

The invention also concerns a method for controlling an anesthesia apparatus of the type with several ventilation modes, in particular, according to the invention, comprising at least one main gas circuit formed by at least one inhalation branch and by at least one exhalation branch, at least one gas accumulation member communicating with the main gas circuit, and relief valve means which have an adjustable pressure set-point level and are arranged on said main gas circuit, in which method:

(a) at least one information item relating to the gas entering and/or leaving the accumulation member is determined,

(b) said at least one information item relating to the gas entering and/or leaving is processed,

(c) at least one information item concerning ventilation mode is deduced from step (b), and

(d) the pressure set-point level is automatically adjusted by acting on said relief valve means as a function of at least the information concerning ventilation mode obtained in step (c).

If appropriate, the method according to the invention can comprise one or more of the following characteristics:

the pressure set-point level is automatically adjusted to a pressure set-point value less than or equal to 5 hPa, preferably less than or equal to 2 hPa, when the information concerning ventilation mode corresponds to information on spontaneous ventilation mode;

the pressure set-point level is automatically adjusted to a pressure set-point value greater than or equal to 5 hPa, preferably between 10 and 90 hPa, when the information concerning ventilation mode corresponds to information on manual ventilation mode;

in step (a), at least one information item relating to the gas entering and/or leaving the accumulation member is determined, chosen from among the pressure of the gas entering and/or leaving the accumulation member, the flowrate of the gas entering and/or leaving the accumulation member, the pressure of the gas inside the accumulation member and/or an information item transmitted by a switch arranged on or in proximity to the connection of the accumulation member.

### BRIEF DESCRIPTION OF THE DRAWING

The present invention will now be described in more detail with reference to the attached figures which are given by way of nonlimiting example and in which:

FIG. 1 shows a diagram of the operating principle of an anesthesia apparatus in spontaneous mode during inhalation by the patient;

FIG. 2 is analogous to FIG. 1 but depicts the spontaneous mode during exhalation by the patient;

FIG. 3 for its part shows the operating principle of the anesthesia apparatus in manual mode during an inhalation phase; and

FIG. 4 shows a diagram of the operating principle of an anesthesia apparatus according to the present invention.

### DESCRIPTION OF THE PREFERRED EMDODIMENTS

FIGS. 1 through 3 show diagrams of an anesthesia apparatus comprising a main gas circuit 1 made up of an inhalation branch 1a and of an exhalation branch 1b, forming a loop circuit or closed circuit.

The downstream part of the inhalation branch 1a is connected to the upper airways of a patient (not shown) via an outlet connection means 2 or connection piece, for example a respiratory mask or an intubation probe, allowing an anesthetic gas to be dispensed to the upper airways of said patient.

In addition, the inhalation branch 1a is connected via its upstream end 3 to a source of anesthetic gases or <<fresh>> gases (not shown), for example a gaseous mixture containing oxygen, nitrogen and halogenated products.

Moreover, the closed circuit 1 also comprises an exhalation branch 1b whose upstream end is connected to the downstream end of the inhalation branch 1a at the connection piece 2, so as to be able to collect the gases exhaled by the patient, and whose downstream end is connected to the upstream end 3 of the inhalation branch 1a so as to constitute the closed circuit 1.

The main circuit 1 also comprises a reservoir balloon 4.

The exhalation branch 1b usually comprises a purification device (not shown) with which it is possible to eliminate at least some of the carbon dioxide (CO₂) contained in the gases exhaled by the patient, for example an adsorbent cartridge.

The exhalation branch 1b also includes an outlet 8 to the atmosphere provided with a relief valve 6 and intended to eliminate any overpressure in the main circuit 1 due in particular to the permanent delivery of fresh gas to the main circuit, the excess being evacuated by the relief valve 6.

In addition, nonreturn valves 7a and 7b are provided on said inhalation branch 1a and exhalation branch 1b, respectively.

The accumulation member 4, here a reservoir balloon, of variable internal volume is connected pneumatically to the main circuit 1 by way of a connection line 9.

As can be seen from FIG. 1, when the patient inhales spontaneously, the gas contained in the accumulation member 4 leaves said accumulation member 4 and is conveyed to the patient via the line 9 and the inhalation branch 1a, being delivered to the patient by way of the connection piece 2.

During the inhalation phase, in spontaneous mode, the gas pressure in the circuit is generally of the order of about 2 hPa.

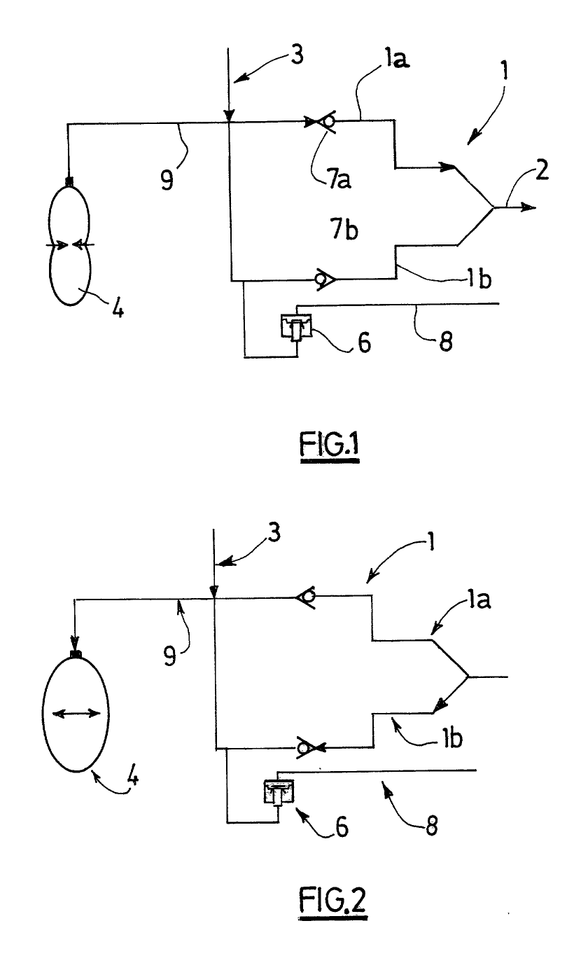

Conversely, as is shown in FIG. 2, during the exhalation phase the patient spontaneously exhales gases rich in CO₂, which gases are first purified by removal of at least some of the CO₂ which they contained, then returned via the lines 1b and 9 to the reservoir balloon 4 where they are stored until the following inhalation phase.

In general, during the exhalation phase, in spontaneous mode, the gas pressure in the circuit is also of the order of about 2 hPa.

In other words, in spontaneous mode, either in the respiration phase (FIG. 1) or exhalation phase (FIG. 2), the control pressure of the relief valve 6 is set at about 2 hPa.

By contrast, as is shown in FIG. 3, in manual mode, during the inhalation phases, it is necessary for the practitioner himself to exert a pressure on the reservoir balloon 4 in order, via the lines 9 and 1a, to expel the accumulated gas and convey it to the patient.

The reason for this is that patients who need to be ventilated manually are those for whom the depth of anesthesia is such that they no longer have automatism in terms of ventilation/respiration, that is to say they cannot independently draw in the gas stored in the reservoir balloon 4.

In manual mode, the gas pressure in the ventilation circuit must therefore be set by the practitioner to a value usually between 10 hPa and 90 hPa, such that the manual pressure exerted by the practitioner on the reservoir balloon 4 in order to expel the gas therefrom does not damage the patient's lungs, while at the same time not thereby inducing inadvertent escape of all the gas via the relief valve 6, which is less resistive than the patient.

To avoid this problem, the control pressure of said relief valve 6 is fixed at a desired value of between 10 and 90 hPa.

In the existing apparatuses, during the exhalation phase, in manual mode, the control pressure does not vary.

Now, from the point of view of safety, it will be readily appreciated that omitting to switch from manual mode to spontaneous mode obliges the patient to force his respiration and to fight against the high pressures in manual mode (10 to 90 hPa), which in some cases can create barotrauma on account of the rise in pressure throughout the ventilator circuit and thus in the patient's lungs.

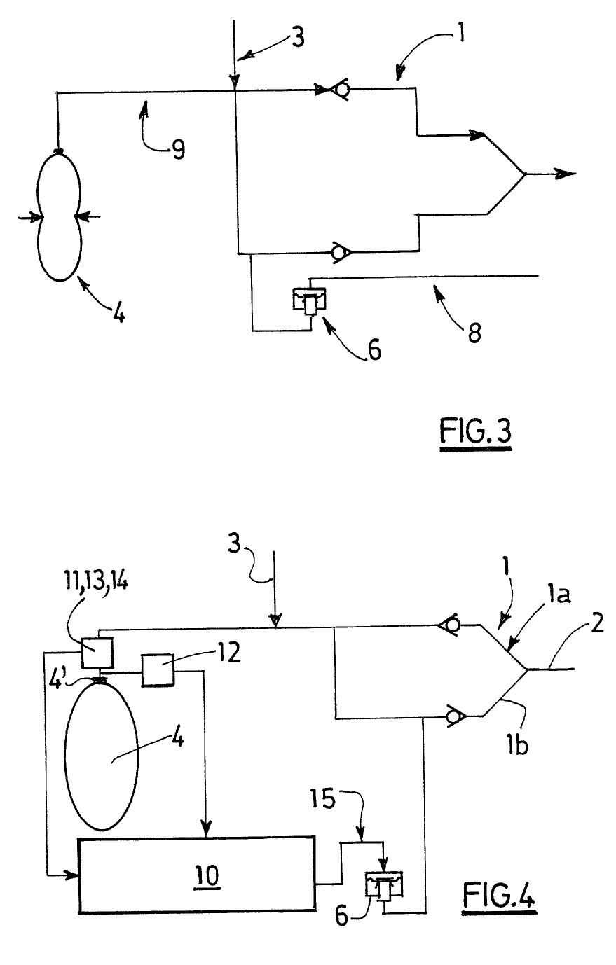

To overcome this problem and to increase patient safety, the present invention proposes an anesthesia apparatus whose operating principle is set out diagrammatically in FIG. 4.

Specifically, to ensure that the patient is not exposed to an overpressure due to the practitioner omitting manual switching upon change-over from a manual ventilation mode to a spontaneous ventilation mode, the anesthesia ventilator according to the invention is provided with detection means, that is to say sensors 11 and 12, connected to or situated on or in immediate proximity to the reservoir balloon 4, for example on the connection line 9 or the connection 4′ linking said connection line 9 to said reservoir balloon 4.

These sensors 11 and 12 make it possible to determine one or more parameters or information items representative of the movements of gas entering and/or leaving the reservoir balloon 4.

After determination of these parameters, they are transmitted to data-processing means 10, such as a processing unit (CPU: central processing unit), where these parameters are processed so as to enable the apparatus to select, in a reliable manner, the mode of ventilation to be observed, depending on whether the movement of gas (outflow of gas) during functioning results from a spontaneous respiratory demand by the patient or, where appropriate, from a maneuver by the practitioner exerting manual pressure on said reservoir balloon.

The function is in fact based on determination of the manual insufflation mode from one or more information items deriving for example from measurement of gas pressure by means of a pressure sensor 12, measurement of gas flowrate by means of a flowrate sensor 11, measurement of a component of the reservoir balloon 4 itself (deformation, etc.), actuation by the practitioner of a switch 13 situated on or in proximity to the reservoir balloon, or retranscription by mechanical means 14 of the pressure prevailing inside said reservoir balloon 4.

In FIG. 4, the ventilator shown comprises a pressure sensor 12 and a flowrate sensor 11 whose measurement parts are situated in immediate proximity to the outlet orifice of the reservoir balloon 4, on the line 9.

In addition, the data-processing means 10 can be a microprocessor system having an analog acquisition chain allowing it to convert the measurements of pressure 12 and/or flowrate 11 into numerical values which, after filtering and numerical correction, can be compared with threshold values making it possible to detect whether the insufflation is imposed by manual actuation by the operator on the reservoir balloon 4 or whether it results from aspiration by the patient.

Moreover, the processing means 10 can, on the basis of the measurements of pressure 12 and/or flowrate 11, determine the characteristics of the reservoir balloon 4, such as its elasticity and its variations in volume, and can determine from this whether the ventilation is manual or spontaneous.

In the case where a switch 13 or a mechanical transmitter 14 is placed in immediate proximity to the connection of the reservoir balloon 4, these transmit directly to the processing means 10 information concerning manual action by the operator on the reservoir balloon 4.

1. An anesthesia apparatus of the type with several ventilation modes, comprising at least:
a main gas circuit (1) formed by at least one inhalation branch (1a) and by at least one exhalation branch (1b),
a gas accumulation member (4) whose internal volume is able to vary and which communicates with said main gas circuit (1),
relief valve means (6) which have an adjustable pressure set-point level and are arranged on said main gas circuit (1),
detection means (11, 12, 13, 14) making it possible to determine at least one information item relating to the gas entering and/or leaving the accumulation member (4),
information-processing means (10) making it possible to process said at least one information item relating to the gas entering and/or leaving in order to deduce from it at least one information item concerning ventilation mode, and
control means (15) acting on the relief valve means (6) in order to automatically adjust the pressure set-point level of said relief valve means (6) as a function of at least the information concerning ventilation mode delivered by said information-processing means (10).

2. The apparatus as claimed in claim 1, wherein said detection means (11, 12, 13, 14) are arranged on or in proximity to the gas accumulation member (4).

3. The apparatus as claimed in either of claims 1 and 2, wherein said detection means (11, 12, 13, 14) are chosen from among:
the sensors (11) for the flowrate of gas entering and/or leaving the gas accumulation member (4),
the sensors (12) for the pressure of gas entering and/or leaving the gas accumulation member (4),
the switches (13) arranged on or in proximity to the connection of the accumulation member (4) and activated directly by the user when he wishes to actuate said relief valve means (6),
the mechanical transmitters (14) making it possible to translate a variation in pressure in the accumulation member (4) into at least one mechanical or electrical information item, and/or
the devices for analyzing the variations in the volume and/or shape and/or pressure of the gas accumulation member (4).

4. The apparatus as claimed in one of claims 1 through 3, wherein the gas accumulation member (4) is a reservoir balloon.

5. The apparatus as claimed in claim 1, wherein the information-processing means (10) comprise at least one microprocessor.

6. The apparatus as claimed in either of claims 1 and 5, wherein the control means (14) act on the relief valve means (6) in order to automatically reduce the pressure set-point level of said relief valve means (6) when the information concerning ventilation mode delivered by the information-processing means (10) corresponds to information on spontaneous ventilation mode.

7. The apparatus as claimed in either of claims 1 and 5, wherein the control means (14) act on the relief valve means (6) in order to automatically increase the pressure set-point level of said relief valve means (6) when the information concerning ventilation mode delivered by the information-processing means (10) corresponds to information on manual ventilation mode.

8. The apparatus as claimed in one of claims 1 through 7, wherein the gas accumulation member (4) and/or the relief valve means (6) are arranged on the exhalation branch (1b) or on the inhalation branch (1a).

9. The apparatus as claimed in one of claims 1 through 7, wherein detection means (11, 12) are arranged on a connection line (9) linking the gas accumulation member (4) to the main gas circuit (1).

10. A method for controlling an anesthesia apparatus of the type with several ventilation modes, in particular as claimed in one of claims 1 through 9, comprising at least one main gas circuit (1) formed by at least one inhalation branch (1a) and by at least one exhalation branch (1b), at least one gas accumulation member (4) communicating with the main gas circuit (1), and relief valve means (6) which have an adjustable pressure set-point level and are arranged on said main gas circuit (1), in which method:
(a) at least one information item relating to the gas entering and/or leaving the accumulation member (4) is determined,
(b) said at least one information item relating to the gas entering and/or leaving is processed,
(c) at least one information item concerning ventilation mode is deduced from step (b), and
(d) the pressure set-point level is automatically adjusted by acting on said relief valve means (6) as a function of at least the information concerning ventilation mode obtained in step (c).

11. The method as claimed in claim 10, wherein the pressure set-point level is automatically adjusted to a pressure set-point value less than or equal to 5 hPa, preferably less than or equal to 2 hPa, when the information concerning ventilation mode corresponds to information on spontaneous ventilation mode.

12. The method as claimed in claim 10, wherein the pressure set-point level is automatically adjusted to a pressure set-point value greater than or equal to 5 hPa, preferably between 10 and 90 hPa, when the information concerning ventilation mode corresponds to information on manual ventilation mode.

13. The method as claimed in claim 10, wherein, in step (a), at least one information item relating to the gas entering and/or leaving the accumulation member (4) is determined, chosen from among the pressure of the gas entering and/or leaving the accumulation member (4), the flowrate of the gas entering and/or leaving the accumulation member (4), the pressure of the gas inside the accumulation member (4) and/or an information item transmitted by a mechanical transmitter or by a switch arranged on or in proximity to the connection of the accumulation member (4).
